(19) United States
(12) Patent Application Publication
(10) Pub. No.: US 2026/0224745 A1
(43) Pub. Date: Aug. 6, 2026

(54) GENE THERAPY FOR TREATING HEARING LOSS DISORDERS

(71) Applicant: OHIO SATE INNOVATION FOUNDATION, Columbus, OH (US)

(72) Inventors: Krystof BANKIEWICZ, Columbus, OH (US); Jerusha NAIDOO, Columbus, OH (US); Yin REN, Columbus, OH (US)

(21) Appl. No.: 19/146,079

(22) PCT Filed: Jan. 10, 2024
(86) PCT No.: PCT/US2024/010999

§ 371 Date: Jul. 7, 2025

Related U.S. Application Data
(60) Provisional application No. 63/479,614, filed on Jan. 12, 2023.

(51) Int. Cl. A61K 48/00; A61K 9/00; A61K 38/17; A61K 38/18; A61P 27/16; C12N 15/86

(52) U.S. Cl. CPC A61K 48/005; A61K 9/0085; A61K 38/1709; A61K 38/185; A61K 48/0075; A61P 27/16; C12N 15/86; C12N 2750/14143

(57) ABSTRACT

Disclosed herein is a method of brainstem delivery of viral vectors in mammals to deliver transgenes to the inner ear via neuronal/axonal transport. This includes delivery of viral vectors to the cochlear nucleus (CN) and superior olivary complex (SOC), areas in the brainstem which send and receive neural inputs to and from the cochlea.

### CROSS-REFERENCE TO RELATED APPLICATIONS

This application claims benefit of U.S. Provisional Application No. 63/479,614, filed Jan. 12, 2023, which is hereby incorporated herein by reference in its entirety.

### BACKGROUND OF THE INVENTION

Hearing loss is the most common sensory impairment affecting nearly 466 million adults and estimated to rise to nearly 1 billion world-wide by 2050 (Lin F R, et al. Neuropsychology. 2011 25(6):763-70). Much of this burden is due to sensorineural hearing loss (SNHL), caused by damage to the sensory cells in the inner ear and auditory neurons connecting the ear to the brain. SNHL is associated with significant social isolation, cognitive impairment (Chern A, et al. Alzheimer Dis Assoc Disord. 2019 33(3):285-90) and tremendous economic burden (Jayakody D M P, et al. Front Neurosci. 2018 12:125). The etiology of SNHL is diverse and includes age-related hearing loss (ARHL), noise-induced hearing loss (NIHL), cochlear synaptopathy, exposure to ototoxic drugs, and genetic hearing loss such as syndromic and non-syndromic deafness (see below for a comprehensive list of examples). Traditional amplification (hearing aids) are the only option for less severe hearing loss, however cochlear implantation (CI) is currently the only FDA-approved device for patients with severe to profound deafness. However, CI does not address the underlying molecular pathophysiology of SNHL. Further, enormous variability exists in CI performance outcomes (Holden L K, et al. Ear Hear. 2013 34(3):342-60; Gifford R H, et al. Audiol Neurootol. 2008 13(3):193-205), much of which is attributed to the varying functional integrity and number of surviving spiral ganglion neurons (SGNs) and their synaptic connections to inner and outer hair cells (IHC/OHCs) in the inner ear (Kamakura T, et al. Hear Res. 2016 339:132-41; Seyyedi M, et al. Otol Neurotol. 2014 35(8):1446-50). These highly specialized neurons are a critical component of the auditory pathway, and in their endogenous state lack regenerative capacity. Therefore, a critical need for a disease-modifying therapy for SNHL remains.

Recent work has focused on gene therapies to deliver genes to the inner ear via delivery of adenovirus (Yagi M, et al. J Assoc Res Otolaryngol JARO. 2000 1(4):315-25; Chikar J A, et al. Hear Res. 2008 245(1-2):24-34; Pfingst B E, et al. J Assoc Res Otolaryngol JARO. 2017 18(6):731-50) and adeno-associated virus (AAV) vectors directly into the cochlea. Intracochlear delivery of AAV can be accomplished via a variety of different surgical approaches (Ren Y, et al. Front Cell Neurosci. 2019 13:323). Direct injection through the round window membrane (RWM) is the most common method, followed by infusion through fenestrating one of the semicircular canals or intratympanic administration (Pfingst B E, et al. J Assoc Res Otolaryngol JARO. 2017 18(6):731-50; Budenz, et al. Sci Rep. 2015 5:8619; Chen H, et al. Gene Ther. 2018 25(4):251-9; Leake P A, et al. J Assoc Res Otolaryngol JARO. 2019 20(4):341-61). Viral-based treatments are a one-time treatment, which achieves sustained gene expression within cells of interest, eliminating the need for a continuous infusion pump or repeated administrations into the cochlea. AAVs delivered directly into brain parenchyma have been shown to be safe and non-pathogenic in multiple human trials for neurological disorders including Clinical Trials NCT01621581, NCT01973543, NCT02022644. It has been established that axonal transport may be harnessed to direct gene expression in brain regions that are not directly injected, the directionality of which is AAV serotype dependent (Salegio E A, et al. Gene Ther. 2013 20(3):348-52). Given the importance of maintaining SGN function and neuronal connecticity to inner ear sensory cells in the treatment of SNHL, an ideal AAV vector and delivery platform for SNHL therapy must transduce SGNs efficiently. However to date, intracochlear delivery of AAVs demonstrate suboptimal SGN transduction efficiency, particularly in the adult mammalian cochlea (Leake P A, et al. J Assoc Res Otolaryngol JARO. 2019 20(4):341-61; Ivanchenko M V, et al. Mol Ther Methods Clin Dev. 2021 21:382-98; Leake P A, et al. Hear Res. 2020 394:107955; Akil O, et al. Hum Gene Ther. 2019 30(1):88-105). For example, Ivachenko et al reported that inner ear delivery of AAV-S led to transduction of SGN cell bodies and fibers in P6 rat pups, but almost no transduction of SGNs in older (P21) rats or juvenile-aged non-human primates (Ivanchenko M V, et al. Mol Ther Methods Clin Dev. 2021 21:382-98). Leake et al reported that only a relatively modest number (approximately 5-10%) of the SGNs expressed GFP following inner ear delivery of AAV2-GFP and AAV5-GFP in 4-week old cats (Leake P A, et al. J Assoc Res Otolaryngol JARO. 2019 20(4):341-61). An ancestral AAV serotype, AAV-Anc80, has been intensively investigated for the delivery of gene delivery to the cochlea. Although the Anc80 vector demonstrated high IHC and OHC transduction following RW injection in neonatal mice (Yoshimura H, et al. Sci Rep. 2018 8(1):2980; Suzuki J, et al. Sci Rep. 2017 7:45524), almost no SGN transduction has been reported in adult rodents (Yoshimura H, et al. Sci Rep. 2018 8(1):2980; Suzuki J, et al. Sci Rep. 2017 7:45524; Richardson R T, et al. Sci Rep. 2021 11(1):11229), gerbils (Richardson R T, et al. Sci Rep. 2021 11(1):11229), or non-human primates (Ivanchenko M V, et al. Hear Res. 2020 394:107930).

With RW delivery, infusate distribution is non-uniform within the cochlear turns (Leake P A, et al. J Assoc Res Otolaryngol JARO. 2019 20(4):341-61), with a gradient of expression highest at the injection site (usually at the cochlear basal turn). Gene delivery to all anatomical regions of the cochlea (apical, middle and basal turns) would allow for more meaningful improvements in auditory thresholds and speech discrimination across the frequency spectrum across low, middle and high frequencies. RW delivery is also known to lead to high artifactual CSF flow and displacement of the drug within minutes (Hirose K, et al. J Assoc Res Otolaryngol JARO. 2014 15(5):707-19), which may necessitate higher viral titers for clinical applications. This could significantly increase treatment cost and lead to non-target delivery such as those reported by Decibel Therapeutics where AAV genomes were detected in the spleen and lymph nodes of non-human primates following intracochlear AAV delivery. Second, much of the initial work has been conducted in young neonatal rodents where displacement of the drug into CSF occurred due to the patency of the cochlear aqueduct at this young age (Akil O, et al. Hum Gene Ther. 2019 30(1):88-105). Third, high levels of transgene overexpression across a multitude of cell types in the inner ear could significantly disturb the system's homeostasis. These types of off-target effects were seen in Leake's study where AAV5-mediated transduction of glial cells that surround the SGN caused significant abnormalities in cellular morphology that was not observed with AAV2, which is neurotropic. (14) Lastly, transduction of antigen presenting glial cells which commonly occurs with RW delivery, with a non-self protein can lead to an undesired immune response (Samaranch L, et al. Mol Ther. 2014 22(2):329-37; Ciesielska A, et al. Mol Ther. 2013 21(1):158-66; Forsayeth J R, et al. Mol Ther J Am Soc Gene Ther. 2011 19(6):1006-7).

### SUMMARY OF THE INVENTION

Disclosed herein is a method of brainstem delivery of viral vectors in mammals to deliver transgenes to the inner ear via neuronal/axonal transport. This includes delivery of viral vectors to the cochlear nucleus (CN) and superior olivary complex (SOC), areas in the brainstem which send and receive neural inputs to and from the cochlea. Axonal transport in this instance describes the movement of viral particles and/or transgenic proteins between the cell body and axon terminals of neurons which innervate the inner ear. Neuronal transport includes transduction of the primary neuron that innervates the cochlea. The disclosed methods cover delivery of AAV particles and resulting transgenic protein to cells and nerve fibers within the mammalian cochlea including but not limited to the spiral ganglion neurons (SGNs), inner and outer hair cells, vestibular (utricle, saccule and semicircular canal) hair cells and all supporting cells.

In a some embodiments, the viral vectors are AAV vectors. By an “AAV vector” is meant a vector derived from an adeno-associated virus serotype, including without limitation, AAV-1, AAV-2, AAV-3, AAV-4, AAV-5, AAV-7, etc. AAV vectors can have one or more of the AAV wild-type genes deleted in whole or part, preferably the rep and/or cap genes, but retain functional flanking ITR Sequences. AAV expression vectors are constructed using known techniques.

Based on the direction of AAV movement, there are two categories of neuronal/axonal transport: anterograde and retrograde neuronal/axonal transport. In anterograde transport, AAV particles are transported from the cell body toward the presynaptic nerve terminals; whereas in retrograde transport, materials are transported from the presynaptic terminals toward the cell body. The disclosed methods include delivery of AAV vectors that undergo both anterograde and/or retrograde neuronal/axonal transport via the afferent and efferent auditory pathways from the cochlear nucleus and superior olivary complex.

The disclosed methods address the pitfalls associated with intracochlear AAV delivery. First, gene delivery is achieved throughout the different cochlear turns (apical, middle and basal) with several commonly used AAV vectors (see FIG. 1). Second, the AAV titer required is significantly reduced compared to RW approaches.

The disclosed methods include transduction of both Type-I and Type-II SGNs, which form the afferent auditory pathway, with AAV vectors. This includes transduction of Type-I SGNs following AAV delivery into the mammalian cochlea nucleus, which are bipolar and have their spiral ganglion cell bodies within the modiolus with dendrites extending to synapse onto inner hair cells and axonal terminals centrally located in the cochlear nuclei of the brainstem. Both anterograde and retrogradely transported vectors may transduce this pathway-bipolar neurons do not have a directionality. The disclosed methods also include transduction of Type-II SGNs following AAV delivery in the cochlea nucleus, which are pseudounipolar nerves that largely innervate the OHCs.

The disclosed methods can involve neuronal/axonal transport to the mammalian cochlea which occurs via multiple synapses (i.e. delivery of transgenes across two synpatic junctions rather than just one, to ultimately get to the sensory cells and not just the nerve fibers). For example, AAV9 is known to traverse multiple synapses. Delivery of an AAV vector to the SOC can deliver genes directly to the OHCs and Type-I SGNs which innervate the IHCs via the inner spiral plexus (a single synapse). When delivered to the SOC, AAV9 may also deliver genes to the IHCs and OHCs via the efferent/retrograde neuronal/axonal transport via retrograde neuronal/axonal transport first to the CN and then to the SGNs which innervate the IHCs and OHCs (two synapses).

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1 shows AAV6-mediated transduction of the rat cochlea following delivery into the brainstem. Maximum intensity projections from confocal z stacks shows GFP expression in nerve fibers at the inner spiral plexus and in nerve fibers innervating OHCs. GFP concentrated in synaptic boutons at the OHC synapse. GFP co-localized with the neurofilament H (NFH) a marker of nerve fibers. Immunohistochemical staining against GFP in the brain demonstrated robust GFP expression in the brainstem covering the cochlea nucleus (panel A) and superior olivary complex (panel B) that did not extend to other brain regions (panel C). High magnification image shows GFP expression in nerve fibers in the cochlea nucleus (panel D).

FIG. 2 shows SGN transduction following AAV6-GFP mediated transduction of rat brainstem. Top panel: GFP-positive cell bodies in the modiolus co-localized with NeuN (red), a marker of neuronal nuclei. Bottom panel: GFP was observed in the auditory nerve, co-localized with neurofilament H (NFH).

FIG. 3 shows infusion of the rat superior olivary complex with AAV2-GDNF led to high levels of GDNF expression locally within the injection site (panel A) and very limited expression in other brain regions (panels B,C). In the cochlea, maximum intensity projections from confocal z stacks revealed high levels of GDNF expression at the synapse with IHCs, the morphology indicated secretion of GDNF from nerve terminals and co-localization with Myosin VIIa shows uptake into IHCs. PBS injected control rat which was imaged with the same confocal laser settings as AAV2-GDNF rat displayed low GDNF levels (bottom row).

FIG. 4 shows AAV-LC.V1-GFP mediated gene transfer in the cochlea following brainstem delivery in the rat. Images show GFP expression in SGN cell bodies in the modiolus and in nerves innervating the inner and outer hair cells (myosin marks inner and outer hair cells).

FIG. 5 shows AAV-Anc80-GFP mediated gene transfer in the cochlea following brainstem delivery in the rat (bottom row shows higher magnification images of hair cell layers). Images show GFP expression in SGNs in the modiolous (top arrow) and inner spiral plexus fibers that innervate IHCS (bottom arrow). Myosin marks inner and outer hair cells.

FIG. 6 shows AAV9-GFP mediated gene transfer to the rat cochlea following brainstem delivery at 1×10¹³ μg/mL. Transduction of high proportion of inner hair cells indicates multiple synaptic neuronal/axonal transport. Native GFP and antibody-mediated detection of GFP transgene are shown. Myosin=inner and outer hair cell marker. NFH (Neurofilament H) is a marker of nerve fibers.

FIG. 7 shows AAV9-GFP mediated gene transfer in the cochlea at 5×10¹² vg/mL (top panel) and 1×10¹¹ μg/mL (bottom panel).

FIG. 8 illustrates infusion into the primate brainstem with convection enhanced delivery under intraoperative MRI-guidance using a SmartFlow cannula and Ball Joint Guide Array (BJGA, Prototek and Technodiamant)). Bilateral frontal curvilinear incision sites are made, and the skull exposed with a retractor (panel A). A burr hole is centered and created over the predefined entry point (Panels B and C). The threaded base is secured with four titanium screws. The BJGA apparatus with a 7-prong MRI contrast-filled fiducial is secured to the base and oriented to the target using stereotactic navigation (panel D). After confirmation of the trajectory to the target, cannulas are placed to the brainstem through the BJGA apparatus (panel E).

FIG. 9 shows quantification of transduction efficiency in the inner spiral plexus, and Type II SGN fiber transduction in the apex, middle and cochlea base following superior olivary complex and cochlea nucleus delivery of various AAV serotypes expressing the reporter gene green fluorescent protein in adult Fisher rats.

FIG. 10 shows quantification of the number of transduced synaptic boutons relative to the number of outer hair cells in the apex, middle and cochlea base following superior olivary complex and cochlea nucleus delivery of various AAV serotypes expressing the reporter gene green fluorescent protein in adult Fisher rats.

FIG. 11 shows quantification of inner hair cell transduction in the apex, middle and base of the cochlea following superior olivary complex and cochlea nucleus delivery of various AAV serotypes expressing the reporter gene green fluorescent protein in adult Fisher rats.

FIG. 12 shows moderate cochlea transduction with AAV2 across cochlea spiral.

FIGS. 13A and 13B show robust cochlea transduction with AAV6 (FIG. 13A) and AAV-Anc80 (FIG. 13B).

FIGS. 14A and 14B show less efficient cochlea transduction with AAV9 and AAV-PhP.B.

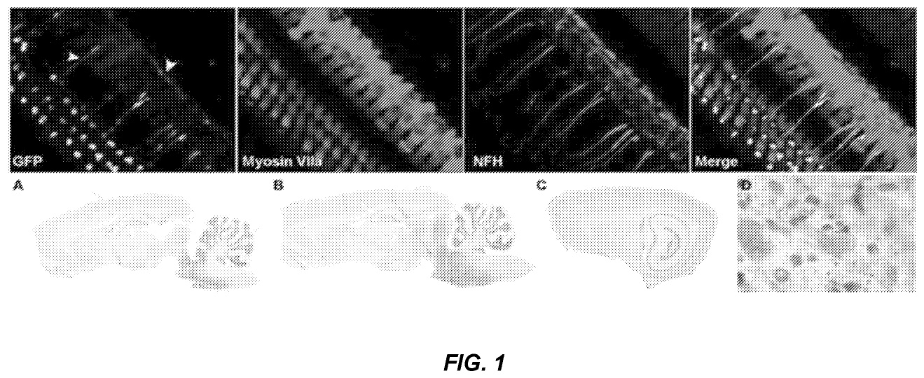

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “gene” refers to a polynucleotide containing at least one open reading frame that is capable of encoding a particular polypeptide or protein after being transcribed or translated.

The term “transgene” as used herein refers to a heterologous gene in a vector.

The term “vector” refers to a replicon into which another DNA segment may be inserted so as to bring about the replication of the inserted segment. The term “expression vector” refers to a vector that includes one or more expression control sequences.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “gene transfer” and “gene delivery” refer to methods or systems for reliably inserting foreign DNA into host cells. Such methods can result in transient expression of non-integrated transferred DNA, extrachromosomal replication and expression of transferred replicons (e.g., episomes), or integration of transferred genetic material into the genomic DNA of host cells. Gene transfer provides a unique approach for the treatment of acquired and inherited diseases. A number of Systems have been developed for gene transfer into mammalian cells. See, e.g., U.S. Pat. No. 5,399,346.

### Viral Vectors

The disclosed therapeutic vector is desirably non-toxic, non-immunogenic, easy to produce, and efficient in protecting and delivering DNA into the target cells. In some embodiments, the viral vector is an adeno-associated virus vector.

More than 30 naturally occurring serotypes of AAV are available. Many natural variants in the AAV capsid exist, allowing identification and use of an AAV with properties specifically suited for the disclosed method. AAV viruses may be engineered by conventional molecular biology techniques, making it possible to optimize these particles for cell specific delivery of RPGR nucleic acid sequences, for minimizing immunogenicity, for tuning stability and particle lifetime, for efficient degradation, for accurate delivery to the nucleus, etc.

Thus, transgene delivery can be achieved by recombinantly engineered AAVs or artificial AAV's that contain sequences encoding the transgene. The use of AAVs is a common mode of exogenous delivery of DNA as it is relatively non-toxic, provides efficient gene transfer, and can be easily optimized for specific purposes. Among the serotypes of AAVs isolated from human or non-human primates (NHP) and well characterized, human serotype 2 is the first AAV that was developed as a gene transfer vector; it has been widely used for efficient gene transfer experiments in different target tissues and animal models. Clinical trials of the experimental application of AAV2 based vectors to some human disease models are in progress, and include such diseases as cystic fibrosis and hemophilia B. Other AAV serotypes include, but are not limited to, AAV1, AAV3, AAV4, AAV5, AAV6, AAV7, AAV8 and AAV9. See, e.g., WO 2005/033321 for a discussion of various AAV serotypes, which is incorporated herein by reference.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the disclosed methods. For illustrative purposes, AAV2/5 is used in the examples described below. In a preferred embodiment, the AAV is AAV2/5. In another preferred embodiment, the AAV is AAV2/8.

In some embodiments, the vectors useful in compositions and methods described herein contain, at a minimum, sequences encoding a selected AAV serotype capsid, e.g., an AAV5 capsid, or a fragment thereof. In another embodiment, useful vectors contain, at a minimum, sequences encoding a selected AAV serotype rep protein, e.g., AAV5 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV5 origin. Alternatively, vectors may be used in which the rep sequences are from an AAV serotype which differs from that which is providing the cap sequences. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector, such as AAV2/8 described in U.S. Pat. No. 7,282,199, which is incorporated by reference herein.

A suitable recombinant adeno-associated virus (AAV) is generated by culturing a host cell which contains a nucleic acid sequence encoding an adeno-associated virus (AAV) serotype capsid protein, or fragment thereof, as defined herein; a functional rep gene; a minigene composed of, at a minimum, AAV inverted terminal repeats (ITRs) and a RPGR nucleic acid sequence; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

Most suitably, such a stable host cell will contain the required component(s) under the control of an inducible promoter. However, the required component(s) may be under the control of a constitutive promoter. Examples of suitable inducible and constitutive promoters are provided herein, in the discussion below of regulatory elements suitable for use with the transgene, i.e., RPGR. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing the rAAV of the disclosed methods may be delivered to the packaging host cell in the form of any genetic element which transfers the sequences carried thereon. The selected genetic element may be delivered by any suitable method, including those described herein. The methods used to construct any embodiment of the disclosed methods are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N. Y. Similarly, methods of generating rAAV virions are well known. See, e.g., K. Fisher et al, 1993 J. Virol., 70:520-532 and U.S. Pat. No. 5,478,745, among others. These publications are incorporated by reference herein.

Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16, or other known and unknown AAV serotypes. These ITRs or other AAV components may be readily isolated using techniques available to those of skill in the art from an AAV serotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

The minigene is composed of, at a minimum, the transgene, regulatory sequences, and 5′ and 3′ AAV inverted terminal repeats (ITRs). In some embodiments, the ITRs of AAV serotype 2 are used. However, ITRs from other suitable serotypes may be selected. It is this minigene which is packaged into a capsid protein and delivered to a selected host cell.

The regulatory sequences include conventional control elements which are operably linked to the transgene in a manner which permits its transcription, translation and/or expression in a cell transfected with the vector or infected with the virus produced by the disclosed methods. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters, are known in the art and may be utilized.

The regulatory sequences useful in the constructs of the disclosed methods may also contain an intron, desirably located between the promoter/enhancer sequence and the gene. One desirable intron sequence is derived from SV-40, and is a 100 bp mini-intron splice donor/splice acceptor referred to as SD-SA. Another suitable sequence includes the woodchuck hepatitis virus post-transcriptional element. (See, e.g., L. Wang and I. Verma, 1999 Proc. Natl. Acad. Sci., USA, 96:3906-3910). PolyA signals may be derived from many suitable species, including, without limitation SV-40, human and bovine.

Another regulatory component of the rAAV useful in the method of the disclosed methods is an internal ribosome entry site (IRES). An IRES sequence, or other suitable systems, may be used to produce more than one polypeptide from a single gene transcript. An IRES (or other suitable sequence) is used to produce a protein that contains more than one polypeptide chain or to express two different proteins from or within the same cell. Preferably, the IRES is located 3′ to the transgene in the rAAV vector.

The selection of the promoter to be employed in the rAAV may be made from among a wide number of constitutive or inducible promoters that can express the selected transgene in the desired an auditory cell. In another embodiment, the promoter is cell-specific. The term “cell-specific” means that the particular promoter selected for the recombinant vector can direct expression of the selected transgene in a particular auditory cell type. In one embodiment, the promoter is specific for expression of the transgene in spiral ganglion neuron cells. In another embodiment, the promoter is specific for expression in the inner and outer hair cells. In another embodiment, the promoter is specific for expression in the inner hair cells. In another embodiment, the promoter is specific for expression in the outer hair cells. In another embodiment, the promoter is specific for expression of the transgene in auditory support cells. In another embodiment, the transgene is expressed in any of the above noted auditory cells.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the chicken β-actin (CBA) promoter, the phosphoglycerol kinase (PGK) promoter, the EF1 promoter (Invitrogen), and the immediate early CMV enhancer coupled with the CBA promoter (Beltran et al, Gene Therapy 2010 cited above).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system; the ecdysone insect promoter, the tetracycline-repressible system, the tetracycline-inducible system, the RU486-inducible system and the rapamycin-inducible system. Other types of inducible promoters which may be useful in this context are those which are regulated by a specific physiological state, e.g., temperature, acute phase, a particular differentiation state of the cell, or in replicating cells only. Any type of inducible promoter which is tightly regulated and is specific for the particular target auditory cell type may be used.

Other regulatory sequences include enhancer sequences. Enhancer sequences useful in the disclosed methods include the IRBP enhancer (Nicord 2007, cited above), immediate early cytomegalovirus enhancer, one derived from an immunoglobulin gene or SV40 enhancer, the cis-acting element identified in the mouse proximal promoter, etc.

Selection of these and other common vector and regulatory elements are conventional and many such sequences are available. See, e.g., Sambrook et al, and references cited therein at, for example, pages 3.18-3.26 and 16.17-16.27 and Ausubel et al., Current Protocols in Molecular Biology, John Wiley & Sons, New York, 1989). Of course, not all vectors and expression control sequences will function equally well to express all of the transgenes.

### Pharmaceutical Compositions and Administration

The recombinant AAV containing the desired transgene and cell-specific promoter for use in the target cells as detailed above is preferably assessed for contamination by conventional methods and then formulated into a pharmaceutical composition intended for parenchymal injection. Such formulation involves the use of a pharmaceutically and/or physiologically acceptable vehicle or carrier, particularly one suitable for administration to the brain, e.g., by subretinal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20.

The composition may be delivered in a volume of from about 50 μL to about 1 mL, including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL.

An effective concentration of a recombinant adeno-associated virus carrying a nucleic acid sequence encoding the desired transgene under the control of the cell-specific promoter sequence desirably ranges between about 10⁸ and 10¹³ vector genomes per milliliter (vg/mL). The rAAV infectious units are measured as described in S. K. Mclaughlin et al, 1988 J. Virol., 62:1963. Preferably, the concentration is from about 1.5×10⁹ vg/mL to about 1.5×10¹² vg/mL, and more preferably from about 1.5×10⁹ vg/mL to about 1.5×10¹¹ vg/mL. In one embodiment, the effective concentration is about 1.5×10¹⁰ vg/mL. In another embodiment, the effective concentration is about 1.5×10¹¹ vg/mL. In another embodiment, the effective concentration is about 2.8×10¹¹ vg/mL. In yet another embodiment, the effective concentration is about 1.5×10¹² vg/mL. In another embodiment, the effective concentration is about 1.5×10¹³ vg/mL. It is desirable that the lowest effective concentration of virus be utilized in order to reduce the risk of undesirable effects, such as toxicity, retinal dysplasia and detachment. Still other dosages in these ranges may be selected by the attending physician, taking into account the physical state of the subject, preferably human, being treated, the age of the subject, the particular ocular disorder and the degree to which the disorder, if progressive, has developed.

### Surgical Delivery Strategy

The AAV vectors can be delivered to the brainstem by any suitable method, for example, injection, grafting, infusion, transplantation of cells carrying the vectors, etc. In a some embodiments, the vectors are delivered by a convection-enhanced delivery (CED) method. CED provides broad, uniform AAV distribution and expression within brainstem nuclei. Any convection-enhanced delivery device may be appropriate for delivery of viral vectors to the mammalian brainstem. In a some embodiments, the device is an osmotic pump or an infusion pump. Both osmotic and infusion pumps are commercially available from a variety of Suppliers, for example Alzet Corporation, Hamilton Corporation, and Aiza, Inc.

In one iteration, Sprague Dawley rats were anesthetized with peritoneal injections of ketamine (5 mg/kg), and they were then maintained under 2% isoflurane for the duration of the surgery in a small-animal stereotactic frame (A) (David Kopf Instruments, Tujunga, CA). The skull was exposed and a burr-hole was created to permit insertion of a cannula (D) for injection of AAV vector expressing the reporter gene green fluorescent protein (GFP) into the brainstem. Animals received a stereotactic injection of 10 μl of AAV-GFP infused at a rate of 1.0 μl/minute via convection enhanced delivery for 10 minutes into the at the following coordinates: AP −3.1 mm, ML +/−2.2 mm, DV −11.7 mm at a 40° angle from the skull base. A customized, stepped (1 mm) silica cannula (C) attached to a 100 μl Hamilton syringe (B) was used for the infusion. This customized cannula reduces reflux along the infusion device by restricting backflow of fluid flow beyond the step (Yin, D; Forsayeth; Bankiewicz K S 2010). To avoid reflux, the cannula was left in place for 2 minutes after infusion.

In some embodiments, AAV vectors can be infused into the primate brainstem with convection enhanced delivery under intraoperative MRI-guidance using a SmartFlow cannula and Ball Joint Guide Array (BJGA, Prototek and Technodiamant)) as described in Bankiewicz et al 2021 (Bankiewicz K S, et al. J Neurosurg. 2021 135(2):651-7). The BJGA allowed for placement of bilateral frontal infusion cannulas. AAV2-hAADC was co-infused with 2 mM of gadoteridol, ProHance (Bracco Diagnostics, Inc.) to allow visualization of vector infusion under intraoperative MRI guidance.

### Treating Hearing Loss

Thee disclosed methods are beneficial for gene delivery in diseases of the inner ear where spiral ganglion neurons and/or inner and outer hair cells of the inner ear are significantly impacted.

In some cases, the disease or indication is “sensorineural hearing loss” defined as broadly, hearing loss caused by damage to: the sensory cells residing in the inner ear (outer hair cells, OHC and inner hair cells, IHC), or auditory nerve fibers and neurons connecting cells in the inner ear to the auditory cortex in the brain.

In some cases, the disease or indication is noise-induced hearing loss, defined as hearing loss due to exposure to excessive noise, including but not limited to: occupational noise exposure, military training or exercises, and noise from social and recreational activities such as firearms. NIHL includes both hearing loss from short exposure to high-intensity noise such as gunfire, fireworks and explosions, or long-term exposure moderate-intensity noise.

In some cases, the disease or indication is ototoxic drug-induced hearing loss caused by drugs including anticancer drugs (including all platinum-based compounds such as cisplatin, carboplatin and oxaloplatin; other anti-cancer compounds including but not limited to methotrexate, vincristine and vinblastine, dactinomycin, bleomycin, nitrogen mustard), antibiotics (including but not limited to aminoglycosides such as gentamicin, macrolides, chloramphenicols, vancomycin, polymyxins, streptomycin, kanamycin, neomycin, amikacin, tobramycin), anti-malarial drugs (quinines), NSAIDs including but not limited to salicylates and naproxen, diuretics (furosemide).

In some cases, the disease or indication involves a combination of ototoxic drugs and noise exposure.

In some cases, the disease or indication is age-related hearing loss-“presbycusis”, the most prevalent sensory deficit in the elderly population affecting 50% of octogenerians and over a third of people over 65 years of age, characterized by difficulty hearing and speech comprehension, especially in noisy environments.

In some cases, the disease or indication is cochlear synaptopathy, aka “hidden hearing loss”, a newly recognized form of hearing loss where there is damage to the synaptic connections between auditory nerve peripheral fibers from SGNs and sensory cells (mainly IHCs), resulting in hearing impairments most noticeable when attempting to hear in noise, despite having a normal audiogram on clinical testing.

In some cases, the disease or indication is a tumor, including but not limited to: vestibular schwannoma, acoustic neuroma, meningioma, facial schwannoma, cochlear schwannoma, epidermoid cyst, endolymphatic sac tumor, hemangioma, paraganglioma, and metastatic tumor.

In some cases, the disease or indication is syndromic deafness (including but not limited to Neurofibromatosis type 2, Waardenburg syndrome, BOR, Usher type I, II, III, Stickler, Pendred, JLNS, Alport).

In some cases, the disease or indication is hearing loss caused by viral and/or bacterial infections including but not limited to meningitis, rubella, CMV, HSV, toxoplasmosis, and S. pneumoniae (Kenneson A, et al. Rev Med Virol. 2007 17(4):253-76).

In some cases, the disease or indication is an auditory neuropathy or synaptopathy—both acquired and congenital/inherited under the umbrella of syndromic and/or non-syndromic (see below).

In some cases, the disease or indication is a monogenetic disorders for example Mutation of OPA1 and Connexin 26 genes (Huang T, et al. Brain Res. 2009 1300:97-104; Kelsell D P, et al. Nature. 1997 387(6628):80-3).

In some cases, the disease or indication is a nonsyndromic deafness including but not limited to the more than 70+ genetic loci identified to date, the most common of which is DFNB1 (GJB2) which accounts for 50% of autosomal recessive hearing loss in certain populations, but also the genes listed in Table 1.

In some cases, the disease or indication is hearing loss associated with mitochondrial diseases for example Mitochondrial Encephalomyopathy with Lactic acidemia and Strokelike episodes (MELAS), Neuropathy or neurogenic muscular weakness with Ataxia and Retinitis Pigmentosa (NARP); Kearns-Sayre syndrome; Pearson Marrow-Pancreas Syndrome; Cytochrome-c Oxidase (complex IV) deficiency (Edmonds, et al. Arch Otolaryngol Neck Surg. 2002 128(4):355).

In some cases, the disease or indication is tinnitus.

In some cases, the disease or indication is an insufficiency and/or variability of speech outcomes after cochlear implantation.

In some cases, the disease or indication is a lysosomal storage disorder associated with sensorineural hearing loss for example Mucopolysaccharidoses Type MPS I, MPS II, MPS III, MPS IVA, and MPS VII (Wolfberg J, et al. Diagn Basel Switz. 2020 10(8):E554).

In some cases, the disease or indication is a neoplasm or other lesions of the cerebellopontine angle (for example metastasis, melanoma, chordoma, lipoma, epidermoid cyst, sarcoidosis).

In some cases, the disease or indication is a radiation induced hearing damage-including but not limited to hearing loss as a consequence of either whole brain irradiation or stereotactic radiosurgery for skull base tumors.

In some cases, the disease or indication is hearing loss as a consequence of diabetic neuropathy.

In some cases, the disease or indication is a hearing impairment in a vascular disorder (Trune D, et al. Semin Hear. 2012 33(03):242-50).

In some cases, the disease or indication is an immune disorders associated with hearing loss.

### Transgenes

The transgene can be any transgene desirable for expression in the inner ear. In some embodiments, the transgene is a neurotrophic factor, such as a Glial Cell Derived Neurotrophic Factor (GDNF). In some embodiments, the transgene is OPA1 or Connexin 26. In some cases, the transgene is a gene from Table 1.

TABLE 1
Autosomal Recessive Nonsyndromic Hearing Impairment:
Genes and Their Clinical Manifestations

| Gene | Locus | Onset | Type |
|---|---|---|---|
| ADCY1 | DFNB44 | Prelingual | Mild to moderate; stable |
| BDP1 | DFNB49 | Postlingual | High frequency; stable |
| BSND | DFNB73 | Prelingual | Severe to profound; stable |
| CABP2 | DFNB93 | Prelingual | Moderate to severe; stable |
| CDC14A | DFNB105 | Prelingual | Severe to profound |
| CDH23 1 | DFNB12 | Prelingual | Severe to profound; stable |
| CIB2 | DFNB48 | Prelingual | Severe to profound |
| CLDN14 | DFNB29 | Prelingual | Severe to profound; stable |
| CLIC5 | DFNB103 | Prelingual | High frequency; progressive |
| COL11A2 | DFNB53 | Prelingual | Severe to profound; stable |
| DCDC2 | DFNB66 | Prelingual | Severe to profound |
| PJVK | DFNB59 | Prelingual | Severe to profound; stable |
| ELMOD3 | DFNB88 | Prelingual | Severe to profound; mixed |
| EPS8 | DFNB102 | Prelingual | Severe to profound |
| EPS8L2 | — | Postlingual | High frequency; progressive |
| ESPN | DFNB36 | Prelingual | — |
| ESRRB | DFNB35 | Unknown | Severe to profound |
| GIPC3 2 | DFNB15/72/95 | Prelingual | Severe to profound |
| GJB2 3 | DFNB1 | Prelingual 4 | Usually stable |
| GJB6 3 | DFNB1 | Prelingual 4 | Usually stable |
| GPSM2 | DFNB32/82 | Prelingual | Severe to profound; stable |
| GRXCR1 | DFNB25 | Prelingual | Moderate to profound; |
| | | | progressive |
| GRXCR2 | DFNB101 | Prelingual | High frequency; progressive |
| HGF | DFNB39 | Prelingual | Severe to profound; |
| | | | downsloping |
| ILDR1 | DFNB42 | Prelingual | Moderate to severe |
| KARS1 | DFNB89 | Prelingual | Moderate to severe; stable |
| LHFPL5 | DFNB67 | Prelingual | Severe to profound; stable |
| LOXHD1 | DFNB77 | Postlingual | Moderate to profound; |
| | | | progressive |
| LRTOMT | DFNB63 | Prelingual | Severe to profound; stable |
| MARVELD2 | DFNB49 | Prelingual | Moderate to profound; stable |
| MET | DFNB97 | Prelingual | Severe to profound |
| MSRB3 | DFNB74 | Prelingual | Severe to profound |
| MYO15A | DFNB3 | Prelingual | Severe to profound; stable |
| MYO3A | DFNB30 | Prelingual | Severe to profound; stable |
| MYO6 | DFNB37 | Prelingual | — |
| MYO7A 5 | DFNB2 | Prelingual, postlingual | Unspecified |
| NARS2 6 | DFNB94 | Prelingual | Severe to profound; stable |
| OTOG | DFNB18B | Prelingual | Mild to moderate; stable |
| OTOGL | DFNB84 | Prelingual | High frequency; stable |
| OTOA | DFNB22 | Prelingual | Severe to profound; stable |
| OTOF | DFNB9 | Prelingual | Usually severe to profound; |
| | | | stable |
| PCDH15 | DFNB23 | Prelingual | Severe to profound; stable |
| PNPT1 | DFNB70 | Prelingual | Severe to profound; stable |
| PTPRQ | DFNB84 | Prelingual | Moderate to profound; |
| | | | progressive |
| RDX | DFNB24 | Prelingual | Severe to profound; stable |
| RIPOR2 | DFNB104 | Prelingual | Severe to profound |
| ROR17 | — | Prelingual | Severe to profound |
| S1PR2 | DFNB68 | Prelingual | Severe to profound |
| SERPINB6 | DFNB91 | Prelingual | Moderate to severe |
| SLC22A4 | DFNB60 | Prelingual | Severe to profound |
| SLC26A4 8 | DFNB4 | Prelingual, postlingual | Stable; progressive |
| SLC26A5 | DFNB61 | Prelingual | Severe to profound; stable |
| STRC | DFNB16 | Prelingual | Severe to profound; stable |
| SYNE4 | DFNB76 | Prelingual | High frequency; progressive |
| TECTA 9, 10 | DFNB21 | Prelingual | Severe to profound; stable |
| TBC1D24 | DFNB86 | Prelingual | Severe to profound |
| TMC1 | DFNB7/11 | Prelingual | Severe to profound; stable |
| TMEM132E | DFNB99 | Prelingual | Severe to profound |
| TMIE | DFNB6 | Prelingual | Severe to profound; stable |
| TMPRSS3 | DFNB8/10 | Postlingual, 11 prelingual | Progressive; stable |
| TPRN | DFNB79 | Prelingual | Severe to profound; stable |
| TRIOBP | DFNB28 | Prelingual | Severe to profound; stable |
| TSPEAR | DFNB98 | Prelingual | Severe to profound |
| USH1C 12 | DFNB18 | Prelingual | Severe to profound; stable |
| WBP2 | — | Prelingual | High frequency; progressive |
| WHRN | DFNB31 | Prelingual | — |

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

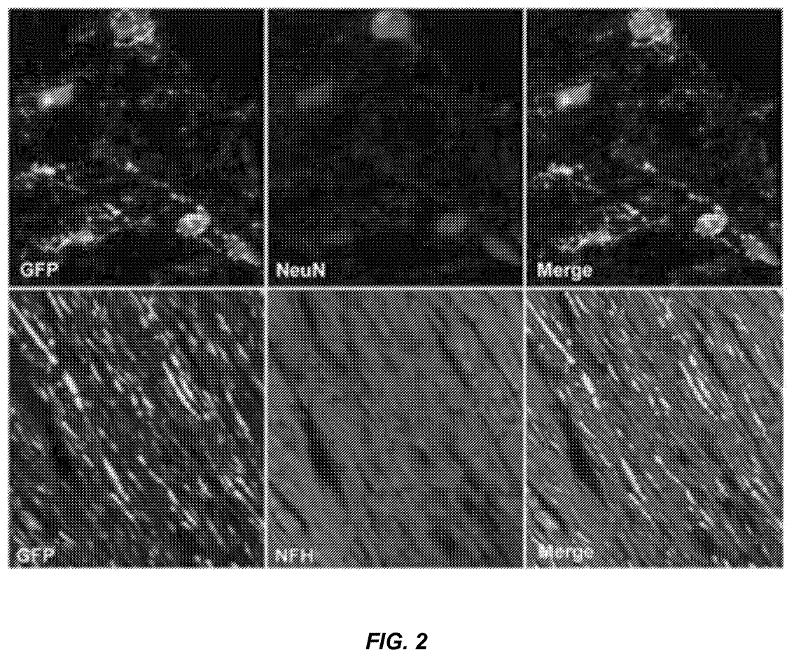

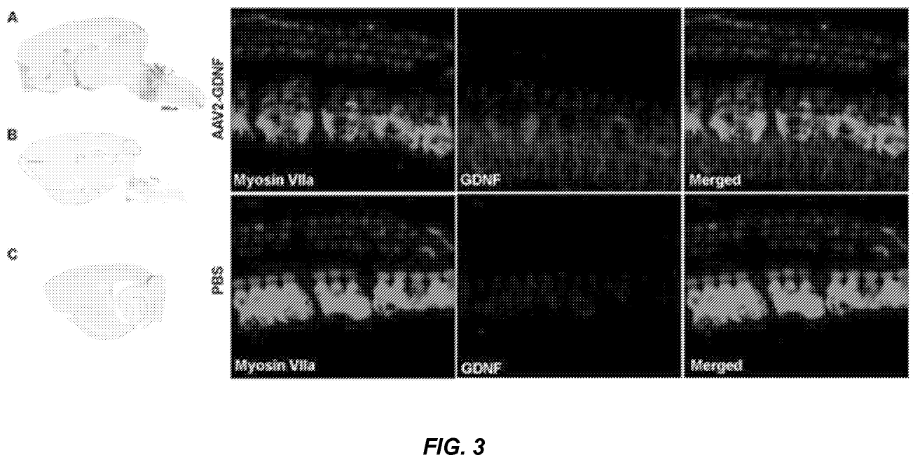

Delivery of an AAV vector which undergoes retrograde neuronal/axonal transport (e.g. AAV6) (Salegio E A, et al. Gene Ther. 2013 20(3):348-52; San Sebastian W, et al. Gene Ther. 2013 20(12):1178-83) to the cochlea nucleus facilitates transduction of SGN afferents, via uptake at nerve terminals in the cochlear nucleus and also lead to transduction SGN cell bodies distally in Rosenthal's canal. Brainstem injection of AAV6-expressing the reporter gene green fluorescent protein (GFP) leads to transduction of nerve fibers in the inner spiral plexus which innervates IHCs and also a high proportion of Type II SGNs that cross the tunnel of corti to innervate OHCs. GFP-positive SGN cell bodies can be observed in the modiolus and GFP-positive fibers were observed in the auditory nerve (FIG. 2) confirming transduction of the entire ascending pathway.

The disclosed methods can also involve transduction of the descending auditory pathways in the mammalian cochlea. This includes AAV-mediated neuronal/axonal transport via both cholinergic feedback systems called the medial and lateral olivocochlear pathways (MOC and LOC) which send signals from the SOC to the OHCs and afferent SGNs, respectively.

### Example 2

Delivery of an AAV vector that undergoes anterograde neuronal/axonal transport (e.g. AAV2) transport to the SOC (where the cell bodies of these pathways reside) would lead to transgene expression in neurons of the lateral and medial olivocochlear pathways, which synapse onto (i) the Type I afferent SGNs and (ii) innervate IHCs, respectively. Delivery of AAV2-encoding Glial Cell Line-Derived Neurotrophic Factor (GDNF) to the brainstem of rats leads to transgenic GDNF expression in inner hair cells via transduction of the SOC and secretion of GDNF from lateral olivocochlear nerves. A high proportion of IHCs within the cochlea mid-region over-express GDNF indicating efficient transduction of the auditory neurons in the LOC pathway. Control rats infused with PBS displayed low levels of basal GDNF expression, consistent with endogenous expression of GDNF in the adult cochlea (Stöver T, et al. Brain Res Mol Brain Res. 2000 76(1):25-35) and the presence of the GDNF co-receptor glycosylphosphatidylinositol (GPI)-linked receptor (GFRa-1) on mature IHCs (Stöver T, et al. Brain Res Mol Brain Res. 2000 76(1):25-35; Ylikoski J, et al. Hear Res. 1998 124(1-2):17-26). GDNF transgene expression was primarily confined to the injection site in the SOC, with very little transgene expression observed in other brain regions. This limited neuronal/axonal transport to other brain regions is very attractive for clinical applications to avoid side effects from off-target expression.

The disclosed methods can also involve brainstem delivery of vectors that undergo both anterograde and retrograde neuronal/axonal transport (e.g. AAV9, AAV-LC.V1). Gene delivery to the mammalian cochlea in the case of these vectors can occur via either the ascending or descending auditory pathways (retrograde and anterograde neuronal/axonal transport), or a mixture of both pathways. The disclosed methods can also involve delivery of bidirectional and unidirectionally transported AAV vectors to the brainstem cochlear nucleus (CN), superior olivary complex (SOC) and a combination of both CN and SOC.

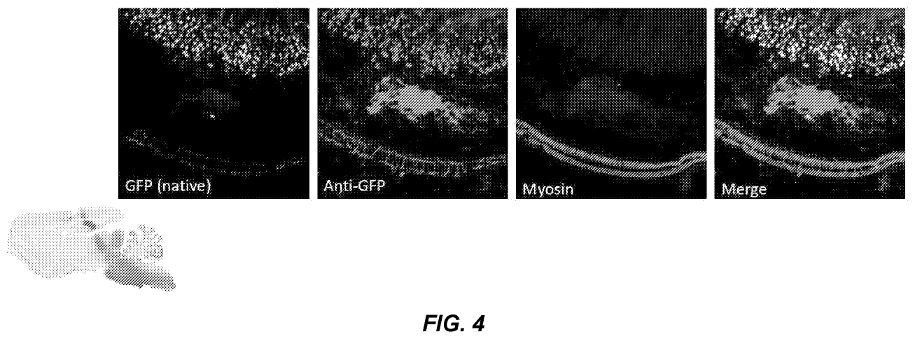

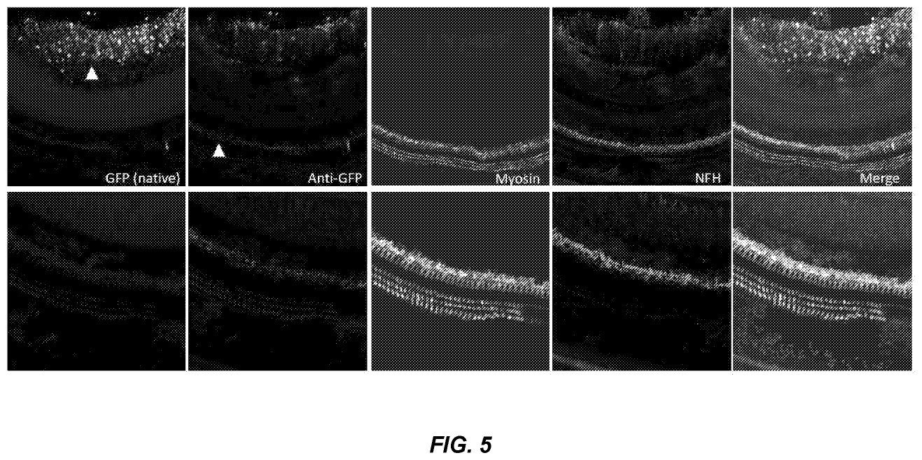

It should be noted that some AAV serotypes preferentially transduce Type I SGNs and IHCs, over Type II SGNs which innervate OHCs. For example, AAV-Anc80 transduces SGN cell bodies within the modiolus and the nerve fibers of the inner spiral plexus which innervates IHCs, but does not transduce SGNs which innervate OHCs (FIG. 5). In contrast AAV-LC.V1 transduces SGN cell bodies in the modiolus as well as the nerve fibers of the inner spiral plexus, and nerve fibers innervating OHCs (FIG. 4).

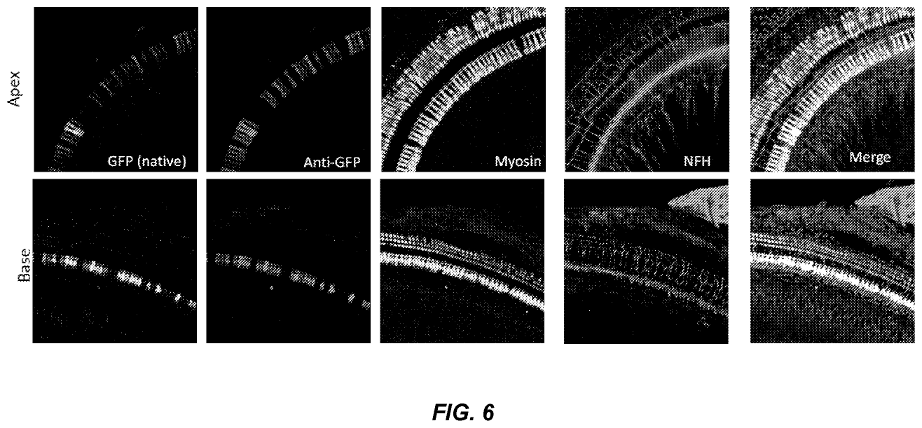

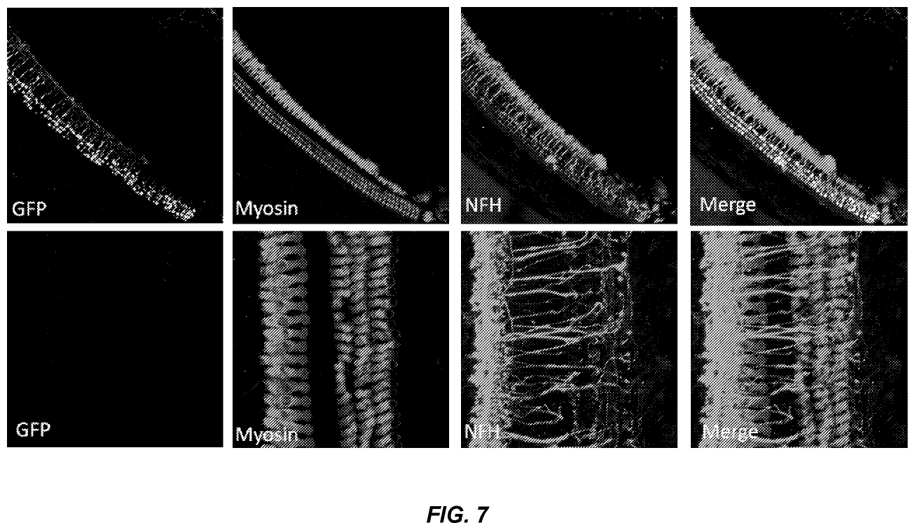

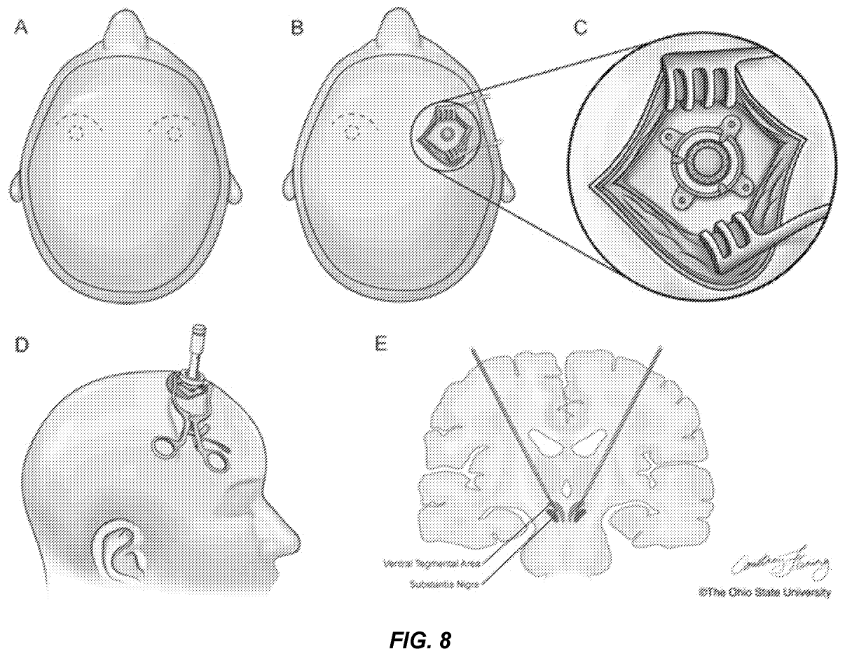

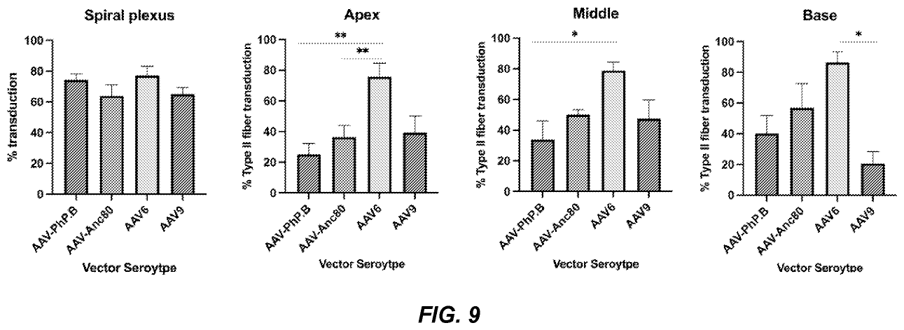

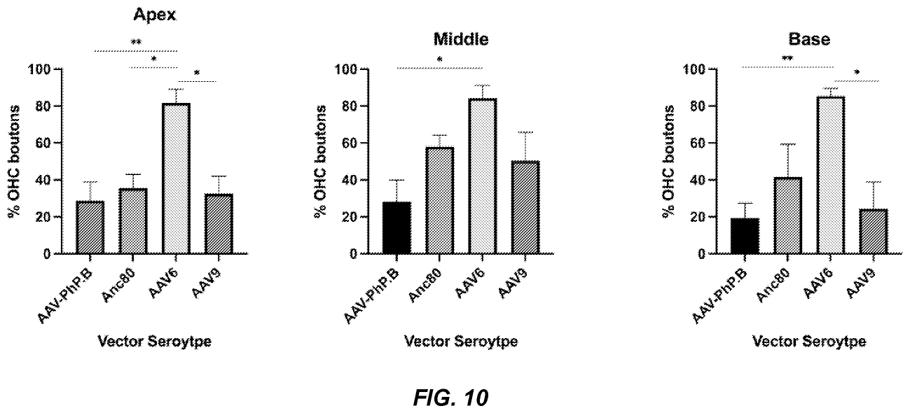

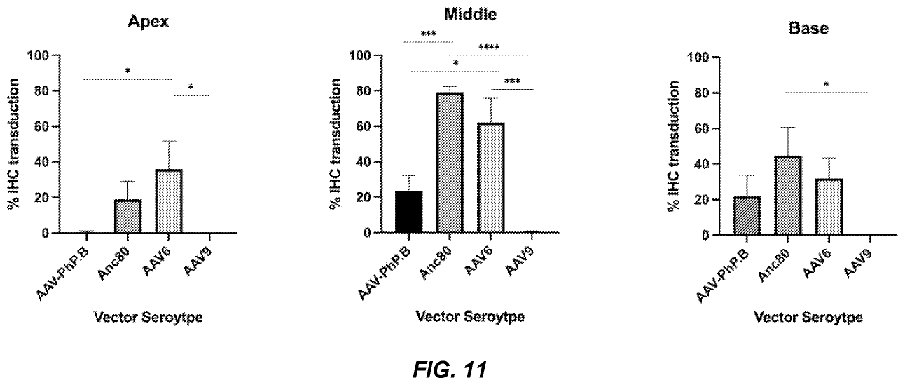

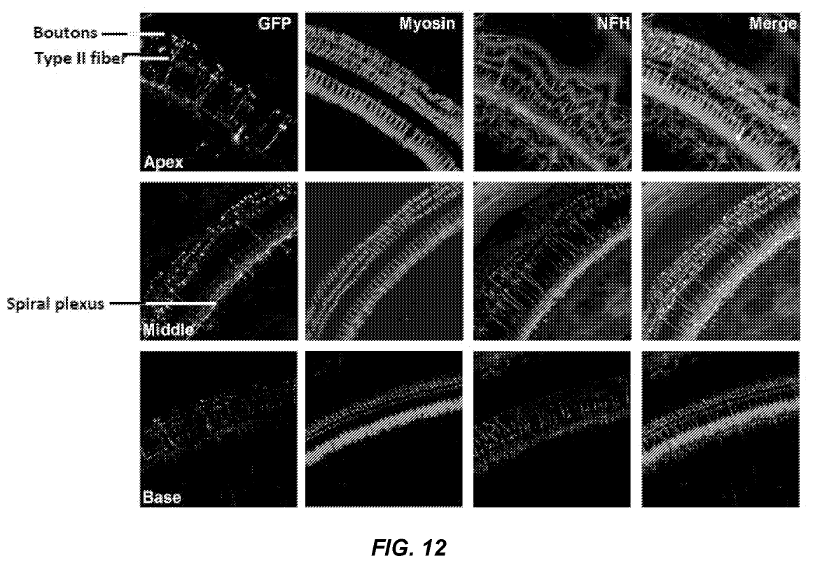

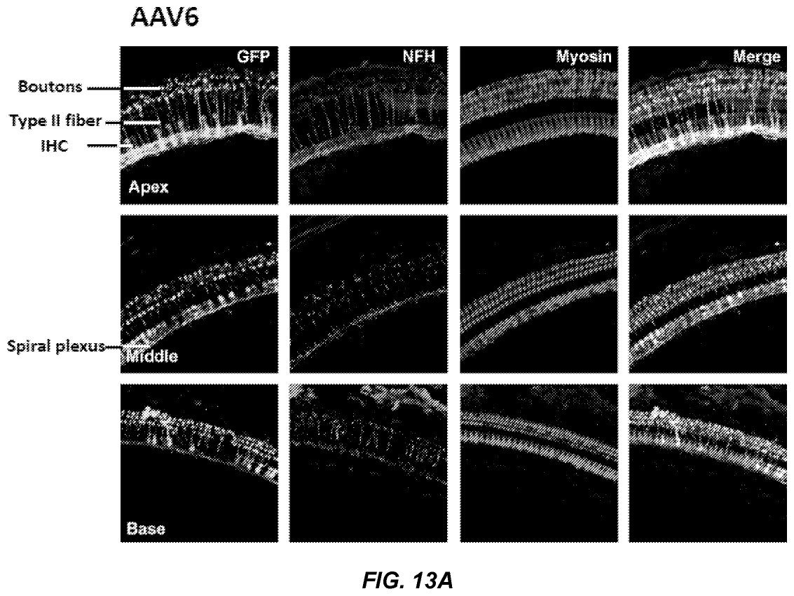

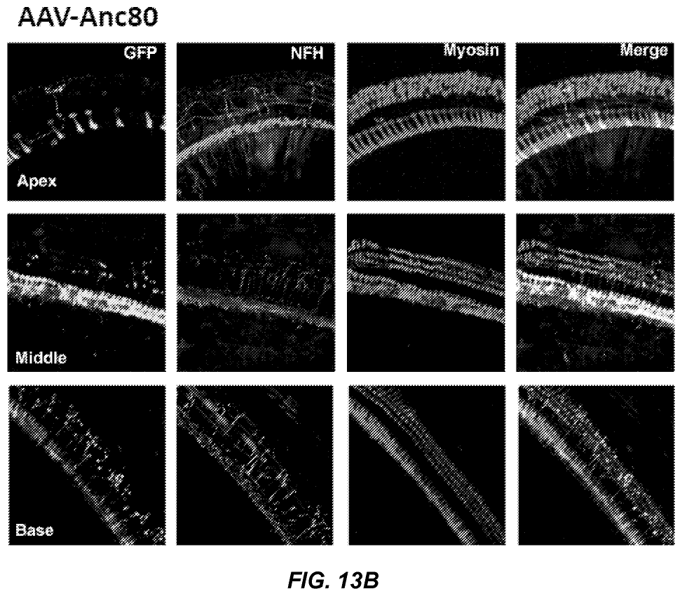

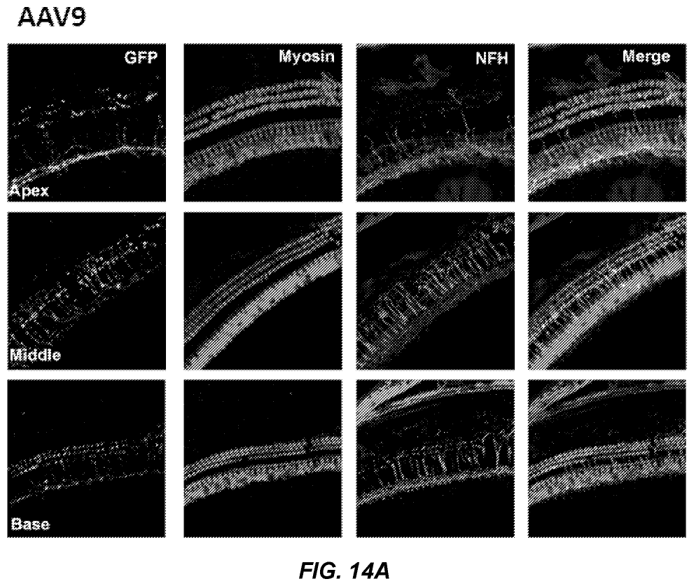

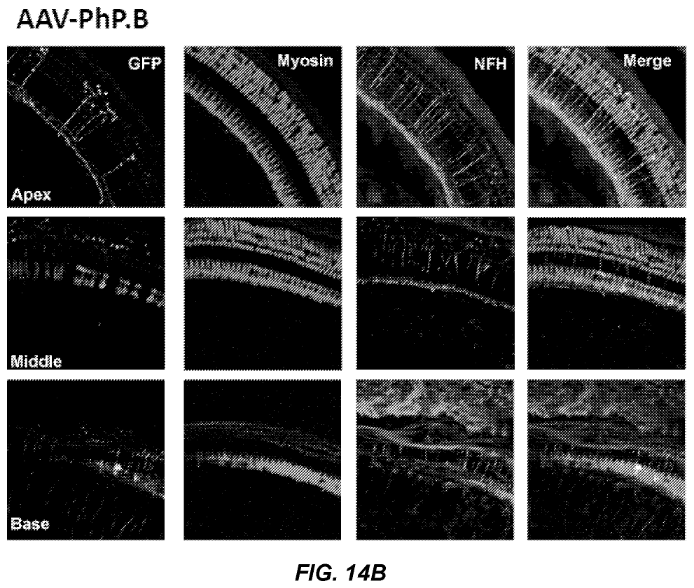

The efficiency of gene transfer and number of synapses traversed by the vector is titer-dependent. This invention covers all possible titers of AAV vectors For example, when AAV9 is injected at high titer of 1.0×10¹³ vg/mL, gene transfer occurs primarily in IHCs, indicative of multi-synaptic neuronal/axonal transport (FIG. 6). When AAV9 is injected at a lower titer of 5.0×10¹² vg/mL, gene transduction occurs primarily in SGN fibers but not hair cells in the cochlea (FIG. 7) indicating fewer synapses are traversed at lower titers. For AAV9, no gene transfer in the cochlea occurs when the titer is lowered to 1×10¹¹ vg/mL.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for delivering a transgene to the inner ear, comprising administering a viral vector encoding the transgene to the cochlear nucleus and/or superior olivary complex of the subject, wherein the viral vector undergoes retrograde neuronal/axonal transport, anterograde neuronal/axonal transport, or a combination thereof.

2. The method of claim 1, wherein the viral vector is an adeno-associated virus (AAV) vector.

3. The method of claim 1, comprising administering a viral vector encoding the transgene to the cochlear nucleus, wherein the viral vector undergoes retrograde neuronal/axonal transport.

4. The method of claim 3, wherein the viral vector is an AAV6 or AAV9 vector.

5. The method of claim 1, comprising administering a viral vector encoding the transgene to the superior olivary complex, wherein the viral vector undergoes anterograde neuronal/axonal transport.

6. The method of claim 5, wherein the viral vector is AAV2 or AAV9.

7. The method of claim 1, wherein the viral vector is administered to the cochlear nucleus and/or superior olivary complex by MRI-guided convection-enhanced delivery (CED).

8. A method for treating hearing loss in a subject, comprising administering a viral vector encoding a transgene that restores hearing to the cochlear nucleus and/or superior olivary complex of the subject.

9. The method of claim 8, wherein the hearing loss is a disease involving spiral ganglion neurons and/or inner and outer hair cells of the inner ear.

10. The method of claim 8, wherein the hearing loss is a sensorineural hearing loss, a noise-induced hearing loss, an ototoxic drug-induced hearing loss, a presbycusis, a cochlear synaptopathy, a syndromic deafness, an auditory neuropathy or synaptopathy, a hearing loss associated with mitochondrial disease, a tinnitus, a hearing loss after cochlear implantation, a hearing loss caused by a lysosomal storage disorder, a radiation-induced hearing damage, a hearing loss from diabetic neuropathy, a hearing impairment from a vascular disorder, or a hearing loss due to an immune disorder.

11. The method of claim 8, wherein the hearing loss is caused by a tumor.

12. The method of claim 8, wherein the hearing loss is caused by a neoplasm or lesions of the cerebellopontine angle.

13. The method of claim 8, wherein the hearing loss is caused by a viral or bacterial infection.

14. The method of claim 8, wherein the hearing loss is a monogenetic disorder.

15. The method of claim 8, wherein the transgene encodes OPA1 and/or Connexin 26.

16. The method of claim 8, wherein the transgene encodes GDNF.

17. The method of claim 8, wherein the transgene encodes a protein selected from the group consisting of ELMOD3, EPS8, EPS8L2, ESPN, ESRRB, GIPC3, GJB2, GJB6, GPSM2, GRXCR1, GRXCR2, HGF, ILDR1, KARS1, LHFPL5, LOXHD1, LRTOMT, MARVELD2, MET, MSRB3, MYO15A, MYO3A, MYO6, MYO7A 5, NARS26, OTOG, OTOGL, OTOA, OTOF, PCDH15, PNPT1, PTPRQ, RDX, RIPOR2, ROR17, S1PR2, SERPINB6, SLC22A4, SLC26A4 8, SLC26A5, STRC, SYNE4, TECTA 9, TECTA 10, TBC1D24, TMC1, TMEM132E, TMIE, TMPRSS3, TPRN, TRIOBP, TSPEAR, USH1C 12, WBP2, and WHRN.
